(19) United States
(12) Patent Application Publication
(10) Pub. No.: US 2023/0042266 A1
(43) Pub. Date: Feb. 9, 2023

(54) NANOBUBBLE-CONTAINING COSMETIC

(71) Applicants: SANSHO KAKEN K.K., Osaka (JP); Nano-Science Laboratory Corporation, Kyoto-shi (JP); H4 Co., Ltd., Osaka (JP)

(72) Inventors: Atsushi Ando, Osaka (JP); Katsuhito Hatanaka, Osaka (JP); Yuji Fujita, Kyoto-shi (JP); Tsuyoshi Kiriishi, Kyoto (JP); Yosuke Takeshima, Osaka (JP)

(73) Assignees: SANSHO KAKEN K.K., Osaka (JP); Nano-Science Laboratory Corporation, Kyoto (JP); H4 Co., Ltd., Osaka (JP)

(21) Appl. No.: 17/863,099

(22) Filed: Jul. 12, 2022

Related U.S. Application Data
(63) Continuation-in-part of application No. PCT/JP2020/001115, filed on Jan. 15, 2020.

(51) Int. Cl. A61K 8/34; A61Q 19/00; B01F 23/20; A61Q 5/00; A61P 9/00

(52) U.S. Cl. CPC A61K 8/34; A61Q 19/00; B01F 23/20; A61Q 5/00; A61P 9/00

(57) ABSTRACT

Provided is a cosmetic comprising nano bubble-liquid and cosmetic ingredients. The cosmetic according to the present invention comprises nano bubble-liquid and cosmetic ingredients, in which the nano bubble-liquid is liquid in a nano bubble state comprising a liquid medium and bubbles, the bubbles having a diameter of 5 nm to 500 nm.

### CROSS REFERENCE

The present application is a continuation-in-part application of a PCT application (PCT/JP2020/001115) designating the United States of America.

The present invention relates to a nano bubble-containing cosmetic. More specifically, it relates to a cosmetic comprising nano bubble-liquid and cosmetic ingredients.

### BACKGROUND OF THE INVENTION

### Field of the Invention

Nano bubble-liquid is liquid having nanobubbles dispersed therein.

Conventionally, a cosmetic containing carbonic acid (carbonic acid and carbon dioxide are used herein with same meaning) is known to have a blood flow enhancement effect when contacted with the skin. Since carbonic acid needs to be stably blended in an aqueous solution in such cosmetic, a method of preparation, for example, by combining two agents at the time of use, is used.

Patent Document 1 describes a liquid cosmetic in which carbonic acid is dissolved, and discloses, as a method to generate carbonic acid, reacting a first agent comprising an acid with a second agent comprising carbonate or bicarbonate to generate carbonic acid.

Non-Patent Document 1 describes characteristics of micro bubbles and nano bubbles and a method of generating the same and the like.

### (Physical Characteristics of Micro Bubbles and Nano Bubbles)

Non-Patent Document 2 reports that micro bubbles and nano bubbles exhibit a diversity that is very sensitive to and moreover greatly dependent on the microscopic bubble creation method as physical properties of.

### (Physiologic Properties of Micro Bubbles and Nano Bubbles)

Non-Patent Document 3 discloses that the physiological characteristics also vary greatly based on the manufacturing method.

Non-Patent Document 4 discloses that micro bubbles generated by a swirling flow system are recognized to have a blood flow enhancement effect. More specifically, it discloses that blood flow enhancement is observed simultaneously upon supply of micro bubbles, while blood flow enhancement ceases upon supply cessation.

On the other hand, swirling flow system bubble generators are also used as nano bubble generators, and it is believed that nano bubbles are generated simultaneously with micro bubbles described in Non-Patent Document 4. Thus, it indirectly discloses that although micro bubbles themselves have a blood flow enhancement effect, nano bubbles that exist after micro bubble supply cessation have no blood flow enhancement effect.

(Circumstances that Necessitate Identification of Nano Bubbles Based on the Manufacturing Method)

As stated above, nano bubbles are known to have greatly varying properties based on the manufacturing method. However, measurement of nano bubbles is accompanied by significant difficulties due to the small bubble diameter of the bubbles and therefore approaches other than the particle diameter and the number concentration generally do not exist.

In particular, the following measurement methods exist:
Particle Tracking Analysis,
Dynamic Light Scattering,
Laser Diffraction and Scattering.
Electrical Sensing Zone Method,
Resonant Mass Measurement, and
Dynamic Image Analysis.

Although these measurement methods can measure the particle diameter and the number concentration, they cannot completely explain the differences in the properties of nano bubbles measured by the generator in practice.

At present, not even a method to directly identify the actual intra-bubble pressure or the molecules present in nano bubbles (whether they are gas molecules or vapor nano bubbles), which can also affect physical properties and bioactivity exists.

Therefore, at the time of filing of the present application, it is difficult to technically and scientifically determine the differences in the properties of nano bubbles based on differences in the manufacturing method, and there is no other way but to identify nano bubbles based on the manufacturing method.

### PRIOR ART DOCUMENTS

### Patent Documents

Patent Document 1: Japanese Patent Application Re-Publication No. 2016/052580

### Non-Patent Documents

Non-Patent Document 1: Hideki TSUGE, “Fundamentals of Microbubbles and Nanobubbles”. Bulletin of the Society of Seawater Science, 2010, P. 4-10, Vol. 64, Issue 1, Japan.
Non-Patent Document 2 Akimi SERIZAWA, “Fundamentals of Micro/Nanobubbles”, Journal of JIME, 2011, P. 861-866, Vol. 46. No. 6, Japan.
Non-Patent Document 3 Hirofumi OHNARI, “Development and Technical Establishment of Micro Bubble Generator and its Technological Establishment”, Nagare 25, 2006, P. 191-195, Japan.
Non-Patent Document 4 Hirofumi OHNARI, “Characteristics and Possibilities of Micro Bubble Technology”, Journal of MMIJ, 2007, P. 89-96, Vol. 123, No. 3, Japan.

### SUMMARY OF THE INVENTION

### Problem to be Solved by the Invention

As stated above, Patent Document 1 discloses, as a method to contain carbonic acid, reacting a first agent comprising an acid with a second agent comprising carbonate or bicarbonate to generate carbonic acid. However, it is difficult to stably contain carbonic acid in an aqueous solution, since the diameter of bubbles increases in the method of preparing a cosmetic containing carbonic acid by combining two agents. Accordingly, there is a problem that a cosmetic containing carbonic acid cannot be stored for a long period of time. In addition, in this method it is difficult to prepare a cosmetic containing gas other than carbonic acid, such as hydrogen, oxygen and air, and a cosmetic containing various types of gas cannot be prepared. Further, there is another problem that the preparation of a cosmetic containing carbonic acid is tedious since two fillers are necessary, and the manufacturing steps increase since it is necessary to manufacture both the first and the second agents, respectively.

Consequently, there is a need for a method of preparing a cosmetic that does not use preparation by combining two agents.

The present invention has been made to solve the problems of the conventional technology described in the above, and provides a cosmetic prepared by dispersing bubbles in a nano bubble state, such as carbonic acid, hydrogen, oxygen, and air, with bubbles having a diameter of 5 nm to 500 nm, into a liquid medium. According to the present invention, since the gas contained in the nano bubble liquid are fine bubbles, the cosmetic can stably contain the gas, and a cosmetic without the risk of gas loss over a long period of time and therefore a manufacturing method thereof can be provided.

### Means for Solving the Problem

The invention according to the first aspect relates to a cosmetic comprising nano bubble-liquid and cosmetic ingredients, in which the nano bubble-liquid is liquid in a nano bubble state comprising a liquid medium and bubbles, the bubbles having a diameter of 5 nm to 500 nm.

The invention according to the second aspect relates to the cosmetic of the first aspect in which the liquid medium is one or more types of liquid media selected from the group consisting of water and ethanol.

The invention according to the third aspect relates to the cosmetic of the first or second aspect, in which nano bubbles are made of one or more types of gas selected from the group consisting of hydrogen, oxygen, carbon dioxide and air.

The invention according to the fourth aspect relates to the cosmetic of any one of the first to third aspects, in which the cosmetic is a skin care cosmetic or a hair cosmetic.

The invention according to the fifth aspect relates to the cosmetic of any one of the first to fourth aspects, in which the cosmetic is for blood flow enhancement.

The invention according to the sixth aspect relates to a method manufacturing of a cosmetic, the method comprising steps of: (a) obtaining nano bubble-liquid with bubbles having a diameter of 5 nm to 500 nm by supplying gas to a liquid medium; and (b) blending the cosmetic ingredients in a liquid medium, in which the steps (a) and (b) are performed in any sequence or simultaneously.

The invention according to the seventh aspect relates to the method of manufacturing a nano bubble-containing cosmetic of the sixth aspect, in which the liquid medium is one or more types of liquid media selected from the group consisting of water and ethanol.

The invention according to the eighth aspect relates to the method of manufacturing a cosmetic of the sixth or seventh aspect, in which the nano bubbles are made of one or more types of gas selected from the group consisting of hydrogen, oxygen, carbon dioxide and air.

The invention according to the ninth aspect relates to the method of manufacturing a cosmetic of any one of the sixth to eighth aspects, in which the cosmetic is a skincare cosmetic or a hair cosmetic.

The invention according to the tenth aspect relates to the method of manufacturing a cosmetic of any one of the sixth to ninth aspects, in which the cosmetic is for blood flow enhancement.

### Effect of the Invention

According to the invention of the first aspect, since a cosmetic comprises nano bubble-liquid and cosmetic ingredients, in which the nano bubble-liquid is liquid in a nano bubble state comprising liquid medium and bubbles, the bubbles having a diameter of 5 nm to 500 nm, the nano bubble-liquid can stably contain gas, and a cosmetic without the risk of gas loss over a long period of time can be provided. Furthermore, since nano bubbles easily permeate into the skin, they have a blood flow enhancement effect, and the cosmetic user can feel warmth after using the cosmetic. In addition, the cosmetic has a moisturizing effect, allowing the cosmetic user to keep the skin moist after using the cosmetic.

According to the invention of the second aspect, since the liquid medium is one or more types of liquid media selected from the group consisting of water and ethanol, the liquid medium can be set within this scope and a suitable cosmetic that suits the cosmetic user can be provided.

According to the invention of the third aspect, since the nano bubble-liquid is in a nano bubble state comprising one or more types of gas selected from the group consisting of hydrogen, oxygen, carbon dioxide and air, the gas can be selected within this scope and a cosmetic with the selected suitable gas that suits the cosmetic user can be provided.

According to the invention of the fourth aspect, since the invention is a skincare cosmetic or a hair cosmetic, a cosmetic in a suitable form can be prepared according to the intended use of the cosmetic user.

According to the invention of the fifth aspect, since the cosmetic is for blood flow enhancement, the cosmetic user can feel warmth after using the cosmetic and a cosmetic with better health benefits can be provided.

According to the invention of the sixth aspect, since the invention comprises steps of (a) obtaining nano bubble-liquid with bubbles having a diameter of 5 nm to 500 nm by supplying gas to a liquid medium; and (b) blending cosmetic ingredients with the liquid medium, in which the steps (a) and (b) are performed in any sequence or simultaneously, the nano bubble-liquid can stably contain gas, and a cosmetic without the risk of gas loss over a long period of time can be provided. Furthermore, since the nano bubbles easily permeate into skin and therefore have a blood flow enhancement effect, the cosmetic user can feel warmth after using the cosmetic. In addition, the method of manufacturing a cosmetic that has a moisturizing effect, allowing the cosmetic user to moisturize the skin after using the cosmetic can be provided.

Furthermore, since the steps (a) and (b) can be performed in any sequence or simultaneously, a suitable manufacturing method can be selected depending on the cosmetic.

According to the invention of the seventh aspect, since the liquid medium can be one or more types of liquid media selected from the group consisting of water and ethanol, the liquid medium can be set within this scope and a suitable cosmetic that suits the cosmetic user can be provided.

According to the invention of the eighth aspect, since the nano bubble-liquid is in a nano bubble state comprising one or more types of gas selected from the group consisting of hydrogen, oxygen, carbon dioxide and air, the gas can be selected within this scope and a cosmetic with the selected suitable gas that suits the cosmetic user can be provided.

According to the invention of the ninth aspect, since the cosmetic is a skincare cosmetic or a hair cosmetic, a cosmetic in a suitable form can be provided according to the intended use of the cosmetic user.

According to the invention of the tenth aspect, since it is a cosmetic for blood flow enhancement, the cosmetic user can feel warmth after using the cosmetic and a cosmetic with better health benefits can be provided.

### DESCRIPTION OF FIGURES

FIG. 1A depicts a histogram of each measurement of the bubble diameter distribution of nano bubbles in nano bubble water prepared by a nanobubble generator (Nano-Science Laboratory Corporation, Model Number “Lab-nano-2004-GN-J-F”) used in an embodiment, at three hours after preparation.

FIG. 1B depicts a histogram of the average of each measurement of the bubble diameter distribution of nano bubbles in nano bubble water prepared by a nano bubble generator (Nano-Science Laboratory Corporation, Model Number “Lab-nano-2004-GN-J-F”), used in one embodiment, at three hours after preparation.

FIG. 2A depicts a histogram of each measurement of the bubble diameter distribution of nano bubbles in nano bubble water prepared by a nano bubble generator (Nano-Science Laboratory Corporation, Model Number “Lab-nano-2004-GN-J-F”), used in one embodiment, at two months after preparation

FIG. 2B depicts a histogram of the average of each measurement of the bubble diameter distribution of nano bubbles in nano bubble water prepared by a nano bubble generator (Nano-Science Laboratory Corporation. Model Number “Lab-nano-2004-GN-J-F”), used in one embodiment, at two months after preparation

### DETAILED DESCRIPTION

Suitable embodiments of the nano bubble-containing cosmetic (hereinafter simply referred to as cosmetic) of the present invention are described below.

The cosmetic of the present invention comprises nano bubble liquid and cosmetic ingredients.

Nano bubble is a generic name for fine bubbles in liquids (mainly water), in which the bubble diameter is less than 1 μm. Further, nano bubbles are also referred to as ultrafine bubbles.

Fine bubbles indicate bubbles with a diameter of 100 μm or less, and are classified into micro bubbles and nano bubbles (ultrafine bubbles). The diameter of micro bubbles ranges from 1 μm to 100 μm, while the diameter of nano bubbles, as described above, is less than 1 μm.

A liquid medium is a medium for supplying gas. Furthermore, it may also be a medium for dissolving or dispersing other ingredients.

Although, for example, water, ethanol, perfumed ethanol, denatured ethanol etc. can be used as liquid media, but are not limited to these, all those obvious to a person skilled in the art can be used, or may be mixed and used.

Water used for a liquid medium can be, but is not limited to, aqua communis, purified water, distilled water, sterile water, saline solution, physiological saline solution etc.

When the liquid medium is water, a cosmetic that a cosmetic user pleasantly can use can be obtained.

A nano bubble liquid is a liquid in which nano bubbles are dispersed.

In a nano bubble liquid, bubbles of any diameter can be used, as long as the diameter of the bubbles is less than 1 μm. For the nano bubble liquid of the present invention, it is desirable to use bubbles with a diameter of 5 nm to 500 nm, and more desirable to use bubbles with a diameter of 50 nm to 500 nm. In addition, an average bubble diameter of the bubbles from 80 nm to 140 nm is desirable.

Apart from that, a lower limit may be 0.1 nm, 1 nm, 3 nm, or may be any value in between. When the bubble diameter is greater than 500 nm, there is a risk of gas loss from the solution, since the bubble diameter is great.

Although, as long as it is a gas, all gasses can be used for nano bubbles contained in nano bubble liquid, using hydrogen, oxygen, carbon dioxide and air is desirable. These gasses can easily permeate into the skin and add a blood flow enhancement effect to a cosmetic.

Ingredients of a cosmetic of the present invention include moisturizers (softeners, emollients), astringents (antiperspirants), cooling agents, whitening agents, UV absorbents, and other agents. Moisturizers can include diglycerin, dipropylene glycol, mixed solution of glycosyl trehalose and hydrogenated starch degradation products, 1,2-pentanediol, 1,2-hexanediol, propanediol, maltitol, sorbitol, glucose, xylitol, 1,2-octanediol, ethylhexylglycerin, sodium acetylated hyaluronate, hydrolyzed hyaluronic acid, hydroxypropyltrimonium hyaluronate, mineral oil, jojoba oil, isopropyl palmitate, isopropyl myristate, tri(caprylic/capric acid) glyceryl, cetyl ethylhexanoate, olive oil, Oryza sativa bran oil, squalane, methyl polysiloxane, decamethylcyclopentasiloxane, lauric acid, myristic acid, palmitic acid, stearic acid, behenic acid, behenyl alcohol, stearyl alcohol, cetyl alcohol, hydrogenated rapeseed oil alcohol, batyl alcohol, arachyl alcohol, and cetearyl alcohol etc. Astringents can include citric acid, lactic acid, aluminium sulfate, lemon water, hamamelis etc. Cooling agents can include menthol, ethyl alcohol, camphor, eucalyptus oil etc. Whitening agents include arbutin, tranexamic acid, retinol etc. UV absorbers include PABA, t-butyl methoxydibenzoylmethane, oxybenzone-1, oxybenzone-2, oxybenzone-3, oxybenzone-4, oxybenzone-5, oxybenzone-6, oxybenzone-9, polysilicone-15, ethylhexyl methoxycinnamate, diethylamino hydroxybenzoyl hexyl benzoate, bis-ethylhexyloxyphenol methoxyphenyl triazine etc. Other agents can include acne agents, dandruff/itching agents, hircismus inhibitor, anti-inflammatory agents (dipotassium glycyrrhizate etc.), disinfectants, nutritional agents, activators and bio-physiological function enhancers etc. However, these are not limited to these examples and all those obvious to a person skilled in the art can be used.

A cosmetic of the present invention may further contain additives, plant extracts and thickeners. Types of additives can include preservatives, antioxidants, metal sequestering agents (metal ion element sequestering agents), anti-fading agents and buffering agents and the like. As for additives, for example, silver oxide, paraoxybenzoates (paraben), sorbic acid, sodium dehydroacetate, quaternary ammonium salts (benzalkonium chloride, benzethonium chloride etc.), chloroxidine, pentylene glycol, and phenoxyethanol can be used as preservatives, tocopherol (vitamin E), ascorbic acid, dibutylhydroxytoluene (BHT), and butyl hydroxyanisole (BHA) can be used as antioxidants, and chelating agents (sodium edetate, ethylenediaminetetraacetate (EDTA), citric acid etc.) can be used as metal sequestering agents, but are not limited to those.

Further, it is desirable to use vitamin extracts and animal extracts (collagen and the like), which can add an excellent blood flow enhancement effect and moisturizing effect to a cosmetic of the present invention.

A cosmetic of the present invention can be prepared as a skincare cosmetic such as lotion, serum, gel, cream, face wash or emulsion and the like. Furthermore, it can also be prepared as a hair cosmetic such as shampoo, rinse, conditioner, treatment, hair tonic and the like. This enables the preparation of a cosmetic in an appropriate form according to the intended use of a cosmetic user. Further, the cosmetic is desirably a lotion. Since the main ingredient of lotion is water, it has low viscosity and there is little risk of gas loss from the nanobubble liquid. This allows it to be used as a lotion with an excellent blood flow enhancement effect.

As stated above, a cosmetic of the present invention comprises nano bubble liquid and has a blood flow enhancement effect, since nano bubbles easily penetrate the skin. It can therefore be used as a cosmetic with a blood flow enhancement effect. This allows a cosmetic user to obtain a sense of warmth after using the cosmetic. The cosmetic also has a moisturizing effect, allowing a cosmetic user to keep the skin moist after using the cosmetic. In addition, a cosmetic user can make the skin smooth after using the cosmetic, in addition to obtaining a feeling of fresh skin.

A method manufacturing the cosmetic of the present invention comprises step (a) obtaining nano bubble liquid with bubbles having a diameter of 5 nm to 500 nm by supplying gas to a liquid medium, and step (b) blending cosmetic ingredients with the liquid medium, in which the steps (a) and (b) can be performed in any sequence or simultaneously.

In step (a), a liquid medium is supplied with gas to obtain a nano bubble-liquid with bubbles having a diameter of 5 nm to 500 nm. More preferably, an average bubble diameter of the bubbles is desirably 80 nm to 140 nm.

As a liquid medium, media stated above, for example, water, ethanol etc. can be used. When the liquid medium is water, a cosmetic that a cosmetic user can pleasantly use can be obtained.

Any means of supplying gas can be used as long as nano bubble liquid can be obtained, but it is desirable to use a nano bubble generator. For example, nano bubble generators made by Nano-Science Laboratory Corporation Ltd can be used as nano bubble generators, but are not limited to those. The rate of gas supply is not specifically limited.

The nano bubble generator used in the present invention is (Nano-Science Laboratory Corporation, Model Number “Lab-nano-2004-GN-J-F”). The generation method and generation principle thereof are a “pore style” that generates nano bubbles only, with little or no gas pressurization. More specifically, it is according to the principles and methods described in WO2020/136716 (applicant Nano-Science Laboratory Corporation).

The nano bubble generator makes it possible to contain nano bubbles with an average diameter of 80 nm to 140 nm and a concentration of 3.0×10⁸-5.0×10⁸ particles/ml, and the device is capable of producing nano bubble liquid with high nanobubble uniformity, high stability and very low contamination of nano particles.

In step b, a liquid medium is blended with cosmetic ingredients.

Cosmetic ingredients can be used as described above.

In the production method of a cosmetic of the present invention, the following patterns are possible, as steps a and b are performed in any order or simultaneously.

A nano bubble liquid obtained by supplying gas to a liquid medium is blended with cosmetic ingredients to obtain a cosmetic (performed in the order of step a, step b).

After blending the cosmetic ingredients with liquid medium, a cosmetic is obtained by supplying gas to obtain nano bubble-liquid (performed in the sequence of step (b), step (a)).

A liquid medium and cosmetic ingredients are blended while supplying gas to obtain a cosmetic (step a and step b are performed simultaneously).

Although all gases that are obvious to a person skilled in the art can be used for supplying gas, hydrogen, oxygen, carbon dioxide and air are desirable. These gases can easily permeate into the skin and add a blood flow enhancement effect to a cosmetic.

A cosmetic manufactured by the manufacturing method described above can be prepared as a skincare cosmetic such as lotion, serum, gel, cream, face wash or emulsion etc. Furthermore, it can also be prepared as a hair cosmetic, such as shampoo, rinse, conditioner, treatment and hair tonic. This enables providing a cosmetic in an appropriate form according to the intended use of a cosmetic user. The cosmetic is desirably a lotion. Since the main ingredient of lotion is water, it has low viscosity and there is little risk of gas loss from the nano bubble solution. This enables manufacturing a lotion with an excellent blood flow enhancement effect.

A cosmetic manufactured by the manufacturing method described above contains nano bubble liquid and has a blood flow enhancement effect, since nano bubbles easily penetrate the skin. As such, it can be manufactured as a cosmetic for blood flow enhancement. Further, the cosmetic also has a moisturizing effect, allowing a cosmetic user to keep the skin moist after using the cosmetic. In addition, a cosmetic user can make the skin smooth after using the cosmetic, in addition to obtaining a feeling of fresh skin.

### EXAMPLES

The following examples of a cosmetic of the present invention makes the effects of the present invention clearer. However, the present invention is not limited to the following examples in any way.

### Example 1

Nano bubble liquid was prepared by aerating purified water with hydrogen gas using a nano bubble generator (Nano-Science Laboratory Corporation, Model Number “Lab-nano-2004-GN-J-F”). The nano bubble liquid and cosmetic ingredients were then blended in the ratio shown in Table 1 and prepared (hereinafter referred to as Example 1). Preparation of the cosmetic and confirming the effect of the cosmetic were performed according to the following. After applying 2 g of a hydrogen-containing cosmetic in which nano-sized gas is dispersed in water to the subject's fingers, blood flow enhancement and capillary dilation were observed using a capillary scope (Kekkan Bijin KK., Product Number SC 10-750) along with measuring the difference in vessel thickness before and after capillary dilation. Further, sensory tests were conducted to determine if there was a difference in sensation in the application area of a subject after applying 5 g of the cosmetic to the entire back of the hand, and sensory tests including the usability of the cosmetic, and the evaluation of the feel of the cosmetic were also performed, and the state of bubbles at one month after preparation of the cosmetic was identified.

TABLE 1
Example 1

| Ingredient Name | Formulation Ratio (%) |
|---|---|
| Water (comprising hydrogen in nano bubble | remainder |
| state) | |
| 1,3-butyleneglycol | 7 |
| Concentrated Glycerin | 5 |
| 1,2-pentanediol | 2 |
| Polyoxyethylene hydrogenated castor oil | 0.1 |
| Phenoxyethanol | 0.5 |

### Example 2

Nano bubble liquid was prepared by aerating purified water with carbon dioxide gas using a nano bubble generator (Nano-Science Laboratory Corporation, Model Number “Lab-nano-2004-GN-J-F”). The nano bubble liquid and cosmetic ingredients were then blended in the ratio shown in Table 2 and prepared (hereinafter referred to as Example 2). Preparation of the cosmetic and confirming the effect of the cosmetic were performed according to the following. After applying 2 g of a carbon dioxide-containing cosmetic in which nano-sized gas is dispersed in water to the subject's fingers, blood flow enhancement and capillary dilation were observed using a capillary scope (Kekkan Bijin KK., Product Number SC 10-750) along with measuring the difference in vessel thickness before and after capillary dilation. Further, sensory tests were conducted to determine if there was a difference in sensation in the application area of a subject after applying 5 g of the cosmetic to the entire back of the hand, and sensory tests including the usability of the cosmetic, and the evaluation of the feel of the cosmetic were also performed, and the state of bubbles at one month after preparation of the cosmetic was identified.

TABLE 2
Example 2

| Ingredient Name | Formulation Ratio (%) |
|---|---|
| Water (comprising carbonic acidin nano bubble | remainder |
| state) | |
| 1,3-butyleneglycol | 7 |
| Concentrated Glycerin | 5 |
| 1,2-pentanediol | 9 |
| Polyoxyethylene hydrogenated castor oil | 0.1 |
| Phenoxyethanol | 0.5 |

### Example 3

Nano bubble liquid was prepared by aerating purified water with oxygen gas using a nano bubble generator (Nano-Science Laboratory Corporation, Model Number “Lab-nano-2004-GN-J-F”). The nano bubble liquid and cosmetic ingredients were then blended in the ratio shown in Table 3 and prepared (hereinafter referred to as Example 3). Preparation of the cosmetic and confirming the effect of the cosmetic were performed according to the following. After applying 2 g of an oxygen-containing cosmetic in which nano-sized gas is dispersed in water to the subject's fingers, blood flow enhancement and capillary dilation were observed using a capillary scope (Kekkan Bijin KK., Product Number SC 10-750) along with measuring the difference in vessel thickness before and after capillary dilation. Further, sensory tests were conducted to determine if there was a difference in sensation in the application area of a subject after applying 5 g of the cosmetic to the entire back of the hand, and sensory tests including the usability of the cosmetic, and the evaluation of the feel of the cosmetic were also performed, and the state of bubbles at one month after preparation of the cosmetic was identified.

TABLE 3
Example 3

| Ingredient Name | Formulation Ratio (%) |
|---|---|
| Water (comprising oxygen in nano bubble | remainder |
| state) | |
| 1,3-butyleneglycol | 7 |
| Concentrated Glycerin | 5 |
| 1,2-pentanediol | 2 |
| Polyoxyethylene hydrogenated castor oil | 0.1 |
| Phenoxyethanol | 0.5 |

### Comparative Example 1

A blank solution was prepared using purified water instead of nano bubble liquid (hereinafter referred to as Comparative Example 1). Confirming the effect of the cosmetic was performed according to the following. After applying 2 g of blank solution to the subject's fingers, blood flow enhancement and capillary dilation were observed using a capillary scope (Kekkan Bijin KK., Product Number SC 10-750) along with measuring the difference in vessel thickness before and after capillary dilation. Further, sensory tests were conducted to determine if there was a difference in sensation in the application area of a subject after applying 5 g of the blank solution to the entire back of the hand, and sensory tests including the usability of the cosmetic, and the evaluation of the feel of the cosmetic were also performed, and the state of bubbles at one month after preparation of the cosmetic was identified.

### Comparative Example 2

A cosmetic was blended in the ratios shown in Table 4 and prepared (hereinafter referred to as Comparative Example 2). Preparation of the cosmetic was performed based on the conventional method of mixing two agents. Further, confirming the effect of the cosmetic was performed according to the following. After applying 2 g of the carbon dioxide-containing cosmetic in which nano-sized gas is dispersed in water to the subject's fingers, blood flow enhancement and capillary dilation were observed and the difference in vessel thickness before and after capillary dilation was measured using a capillary scope (Kekkan Bijin KK., Product Number SC 10-750). Further, sensory tests were conducted to determine if there was a difference in sensation in the application area of a subject after applying 5 g of the cosmetic to the entire back of the hand, and sensory tests including the usability of the cosmetic, and the evaluation of the feel of the cosmetic were also performed, and the state of bubbles at one month after preparation of the cosmetic was identified.

TABLE 4
Comparative Example 2

| | Formulation Ratio (%) |
|---|---|
| Agent 1 | |
| Water | remainder |
| Concentrated Glycerin | 8 |
| Sodium Hydrogen Carbonate | 5 |
| Tamarind Seed Gum | 3.5 |
| Phenoxyethanol | 0.5 |
| Agent 2 | |
| Concentrated Glycerin | 92 |
| Citric Acid | 8 |

### Comparative Example 3

Milli bubble liquid was prepared by aerating purified water with hydrogen gas using a cylinder. The milli bubble liquid and cosmetic ingredients were then blended in the ratio shown in Table 5 and prepared (hereinafter referred to as Comparative Example 3). Further, confirming the effect of the cosmetic was performed according to the following. After applying 2 g of the milli bubble liquid hydrogen to the subject's fingers, blood flow enhancement and capillary dilation were observed using a capillary scope (Kekkan Bijin KK., Product Number SC 10-750) along with measuring the difference in vessel thickness before and after capillary dilation. Further, sensory tests were conducted to determine if there was a difference in sensation in the application area of a subject after applying 5 g of the cosmetic to the entire back of the hand, and the usability of the cosmetic, evaluation of the feel of the cosmetic, and the state of bubbles at one month after preparation of the cosmetic were identified.

TABLE 5
Comparative Example 3

| Ingredient Name | Formulation Ratio (%) |
|---|---|
| Water (comprising hydrogen) | remainder |
| 1,3-Butylene glycol | 7 |
| Concentrated Glycerin | 5 |
| 1,2-Pentanediol | 2 |
| Polyoxyethylene hydrogenated castor oil | 0.1 |
| Phenoxyethanol | 0.5 |

### Comparative Example 4

Milli bubble solution was prepared by aerating purified water with carbon dioxide gas using a cylinder. The milli bubble solution was then blended and prepared in the ratio shown in Table 6 (hereinafter referred to as Comparative Example 4). Confirming the effect of the cosmetic was performed according to the following. After applying 2 g of a carbon dioxide comprising cosmetic in which gas is dispersed in water to the subject's fingers, blood flow enhancement and capillary dilation were observed and the difference in vessel thickness before and after capillary dilation was measured was measured using a capillary scope (Kekkan Bijin KK., Product Number SC 10-750). Further, sensory tests were conducted to determine if there was a difference in sensation in the application area of a subject after applying 5 g of the blank solution to the entire back of the hand, and sensory tests including the usability of the cosmetic, and the evaluation of the feel of the cosmetic were also performed, and the state of bubbles at one month after preparation of the cosmetic was identified.

TABLE 6
Comparative Example 4

| Ingredient Name | Formulation Ratio (%) |
|---|---|
| Water (comprising carbonic acid) | remainder |
| 1,3-Butylene glycol | 7 |
| Concentrated Glycerin | 5 |
| 1,2-Pentanediol | 2 |
| Polyoxyethylene hydrogenated castor oil | 0.1 |
| Phenoxyethanol | 0.5 |

### Comparative Example 5

Milli bubble solution was prepared by aerating purified water with oxygen gas using a cylinder. The milli bubble solution was then blended and prepared in the ratio shown in Table 7 (hereinafter referred to as Comparative Example 5). Confirming the effect of the cosmetic was performed according to the following. After applying 2 g of an oxygen-containing cosmetic in which gas is dispersed in water to the subject's fingers, blood flow enhancement and capillary dilation were observed and the difference in vessel thickness before and after capillary dilation was measured using a capillary scope (Kekkan Bijin KK., Product Number SC 10-750). Further, sensory tests were conducted to determine if there was a difference in sensation in the application area of a subject after applying 5 g of the cosmetic to the entire back of the hand, and sensory tests including the usability of the cosmetic, the evaluation of the feel of the cosmetic were also performed, and the state of bubbles at one month after preparation of the cosmetic was identified.

TABLE 7
Comparative Example 5

| Ingredient Name | Formulation Ratio (%) |
|---|---|
| Water (comprising oxygen) | remainder |
| 1,3-Butylene glycol | 7 |
| Concentrated Glycerin | 5 |
| 1,2-Pentanediol | 2 |
| Polyoxyethylene hydrogenated castor oil | 0.1 |
| Phenoxyethanol | 0.5 |

After the cosmetic was applied to the subject's fingers, capillary dilation was observed using a capillary scope (Kekkan Bijin KK., Product Number SC 10-750), and the blood flow enhancement effect was measured based on the criteria in Table 8.

TABLE 8
Criteria for Blood Flow-Promoting Effect

| Symbol | Effect |
|---|---|
| ⊚ | Blood Flow-Promotion and Capillary Dilation is Observed |
| ◯ | Either Blood Flow Promotion or Capillary Dilation is Observed |
| Δ | Either Slight Blood Flow Promotion or Capillary Dilation |
| | is Observed |
| X | No Blood Flow Promotion or Capillary Dilation is Observed |

Bubble diameter was measured using NanoSight LM10V-HS/England, Malvem.

After the cosmetic was applied to the entire back of the hand, a sensory test was performed for difference in sensation in the application area of the subject.

TABLE 9
Criteria for Differences in Sensation in Application Area

| Symbol | Effect |
|---|---|
| ⊚ | Able to Feel Blood Flow-Promoting Effects such as Sense |
| | of Warmth, Redness of Skin etc. |
| ◯ | Able to Feel Blood Flow-Promoting Effects such as either |
| | Sense of Warmth, or Redness of Skin etc, |
| Δ | Able to Feel Blood Flow-Promoting Effects such as Slight |
| | Sense of Warmth, Slight Redness of Skin etc. |
| X | Not Able to Feel Blood Flow-Promoting Effects such as Sense |
| | of Warmth, Redness of Skin etc. |

For determining the bubble condition of a cosmetic at one month after preparation, each cosmetic was allowed to stand at room temperature for one month after preparation and exposed to light in a glass container, and then, the bubble condition was visually confirmed and assessed based on the criteria in Table 10.

The bubble condition can also be assessed by other measurement methods, for example, by measuring the number of bubbles in the cosmetic and comparing the values. 3 and comparing the values.

TABLE 10
State of Bubbles at One Month after Manufacture of Cosmetic

| Symbol | Effect |
|---|---|
| ⊚ | Fully Comprises Bubbles |
| ◯ | Comprises Bubbles |
| Δ | Hardly Comprises Bubbles |
| X | Comprises no Bubbles |

TABLE 11
Test Results

| | | | | Comparative | Comparative | Comparative | Comparative | Comparative |
|---|---|---|---|---|---|---|---|---|
| | Example 1 | Example 2 | Example 3 | Example 1 | Example 2 | Example 3 | Example 4 | Exampie 5 |
| Bubble Diameter | 5 nm- | 5 nm- | 5 nm- | — | 1 mm- | 1 mm- | 1 mm- | 1 mm- |
| | 500 nm | 500 nm | 500 nm | | 7.5 mm | 30 mm | 30 mm | 30 mm |
| Difference in | 0.7 mm | 0.5 mm | 0.3 mm | 0.1 mm | 0.6 mm | 0.1 mm | 0.1 mm | 0.1 mm |
| Vessel Thickness | | | | or less | | or less | or less | or less |
| Before and After | | | | | | | | |
| Capillar/Dilation | | | | | | | | |
| Blood Flow | ⊚ | ⊚ | ◯ | X | ⊚ | X | X | X |
| Ehancement Effect | | | | | | | | |
| Difference in | ⊚ | ⊚ | Δ | X | ⊚ | X | X | X |
| Sensation in the | | | | | | | | |
| Application Area | | | | | | | | |
| Usability | ◯ | ◯ | ◯ | ◯ | X | ◯ | ◯ | ◯ |
| Asessment of | When | When | When | When | When | When | When | When |
| Sense in Using | Applied, | Applied, | Applied, | Applied, | Applied, | Applied, | Applied, | Applied, |
| the Cosmetic | Moist | Moist | Moist | Moist | Foam and | Moist | Moist | Moist |
| | Feeling and | Feeling and | Feeling and | Feeling | Moist | Feeling | Feeling | Feeling |
| | Better | Better | Better | | Feeling on | | | |
| | Fitness to | Fitness to | Fitness to | | the Skin | | | |
| | the Skin | the Skin | the Skin | | | | | |
| State of Bubble | ⊚ | ⊚ | ⊚ | X | X | X | X | X |
| at One Month | | | | | | | | |
| after Manufacture | | | | | | | | |
| of Cosmetic | | | | | | | | |

The test results of the cosmetics in Examples 1 to 3 and Comparative Examples 1 to 5 shown in Table 11 will be discussed below.

In Examples 1 to 3, the difference in vessel thickness before and after capillary dilation was 0.7 mm. 0.5 mm, and 0.3 mm respectively, which indicates a great increase in thickness. On the other hand, in Comparative Example 1 in which purified water was used as a blank, the difference in vessel thickness before and after capillary dilation was 0.1 mm or less and no change was observed. This showed that the nano bubble-containing cosmetics in Examples 1-3 have blood flow-promoting effects.

In Comparative Example 2, a carbonic acid-containing cosmetic was prepared using the conventional method of mixing two agents. When the effect of the cosmetic was confirmed, the difference in blood vessel thickness before and after capillary dilation was 0.6 mm, which indicates that the carbonic acid-containing cosmetic of Comparative Example 2 has a blood flow-promoting effect.

In Comparative Examples 3 to 5, gas-containing cosmetics were prepared by aerating gas into purified water using cylinders. When the effects of the cosmetics were confirmed, the differences in blood vessel thickness before and after capillary dilation were 0.1 mm or less, which indicates that the cosmetics in Comparative Examples 3 to 5 do not have a blood flow enhancement effect. Further, since the diameters of the bubbles contained in the cosmetics of Examples 1 to 3 were from 5 nm to 500 nm, and the diameters of the bubbles contained in the cosmetics of Comparative Examples 3 to 5 were from 1 mm to 30 mm, it was shown that the cosmetics with nano bubble liquid have blood flow enhancement effects. On the other hand, even if the diameter of the bubble was between 1 mm and 7.5 mm as in Comparative Example 2, it was shown that the cosmetic, with bubble-liquid prepared when needed, has a blood flow enhancement effect.

Further, the state of bubbles at one month after preparation of the cosmetic was confirmed. The nano bubble-containing cosmetics in Examples 1 to 3 were shown to fully comprise bubbles even at one month after preparation of the cosmetics. The results show that the nano bubble-containing cosmetics in Examples 1 to 3 are cosmetics having no risk of gas loss over a long period of time. On the other hand, the cosmetics in Comparative Examples 1 to 5 were shown to contain no bubbles at one month after preparation. This indicates that the gas is quickly lost from the cosmetic due to the large diameter of the bubbles contained the cosmetics in Comparative Examples 1 to 5. Thus, it was shown that the nano bubble-containing cosmetics in Examples 1 to 3 can be stored for a longer period of time and are more stable when compared to the cosmetics in Comparative Examples 1 to 5.

### Discussion

The nano bubble-containing cosmetics in Examples 1 to 3 can be prepared more easily than the carbonic acid-containing cosmetic in Comparative Example 2, prepared by the conventional method of mixing two agents. Further, since gas can be selected from the group consisting of hydrogen, carbonic acid and oxygen, a cosmetic that suits the cosmetic user can be used. Additionally, since a nano bubble-containing cosmetic uses nano bubble liquid having a small bubble diameter, it is possible to provide a cosmetic in which gas loss is less than, for example, the carbonic acid-containing cosmetic of Comparative Example 2, and which has no risk of gas loss over a long period of time can be prepared. Further, since Comparative Example 2 requires the cosmetic to be used immediately after combining the two agents, it has low usability as a cosmetic. Thus, the nano bubble-containing cosmetics in Examples 1 to 3 are superior to the cosmetic in Comparative Example 2 in terms of usability as a cosmetic.

### Example 4

Nano bubble liquid was prepared by aerating purified water with air using a nano bubble generator (Nano-Science Laboratory Corporation, Model Number “Lab-nano-2004-GN-J-F”). The nano bubble-liquid was then stored at room temperature (20° C.-25° C.) and nano bubble diameter and concentration were measured at the time points described in Table 12 using a nano particle measuring device manufactured by NanoSight LM10V-HS/England, Malvem.

A nano bubble generator (Nano-Science Laboratory Corporation, Model Number “Lab-nano-2004-GN-J-F”) could make water contain nanobubbles with an average diameter of 80 nm to 140 nm and a concentration of 3.0×10⁸-5.0×10⁸ particles/ml at any time points in Table 12. Now the bubble diameter distribution at 3 hours and 2 months after manufacturing is disclosed.

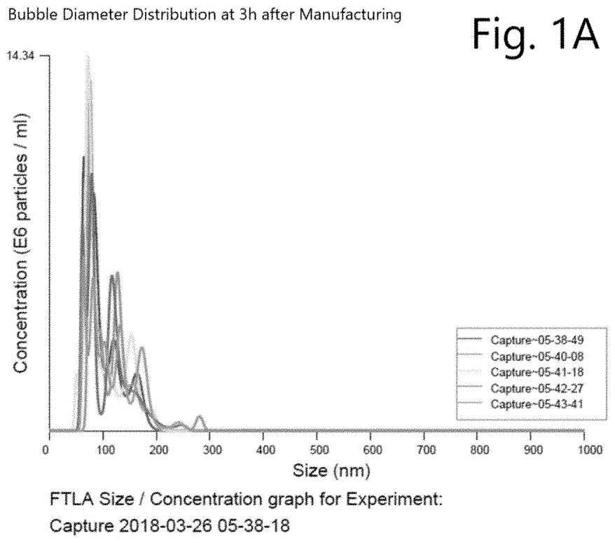

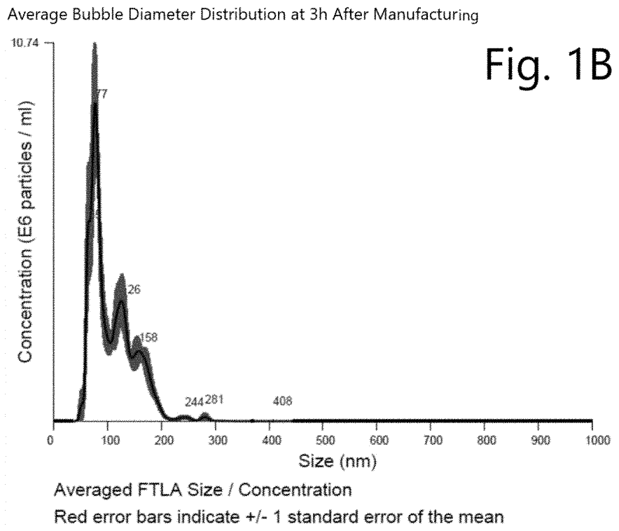

A histogram of each measurement of the bubble diameter distribution of nano bubbles at 3 hours after manufacturing is shown in FIG. 1A, and a histogram of the average of each measurement of the bubble diameter distributions of nano bubbles at 3 hours after manufacturing is shown in FIG. 1B.

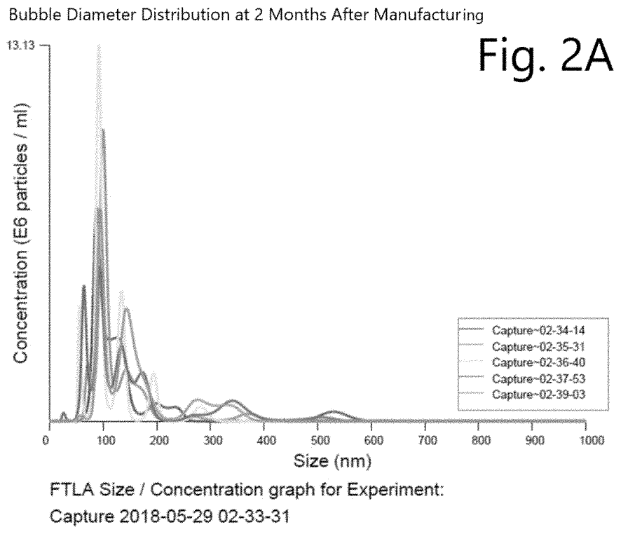

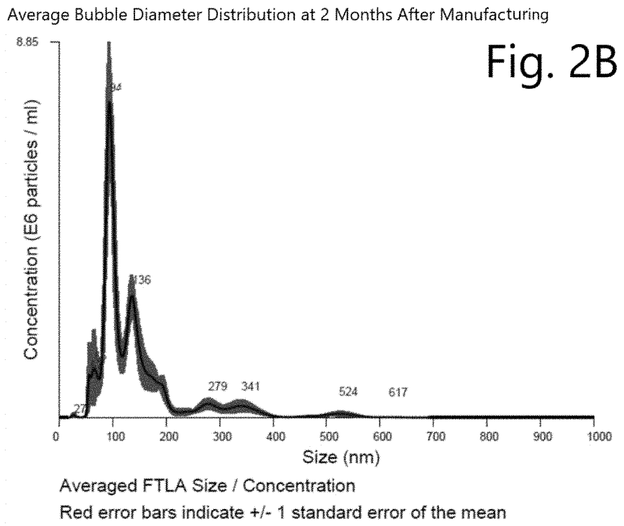

A histogram of each measurement of the bubble diameter distributions of nano bubbles at 2 months after manufacturing is shown in FIG. 2A, and a histogram of the average of each measurement of the bubble diameter distributions of nano bubbles at two months after manufacturing is shown in FIG. 2B.

It can be understood from FIGS. 1A, 1B, 2A and 2B that preparing nano bubbles liquid can make water contain nano bubbles with an average diameter of 80 to 140 nm and a concentration of 3.0×108-5.0×108 particles/ml for at least 2 months after preparation.

TABLE 12

| | | | | | | |
|---|---|---|---|---|---|---|
| Condition | Immediately After | 1 Day | 3 Days | 3 Weeks | 1 Month | 2 Months |

| | |
|---|---|
| Gas | Air |
| Medium | Water |
| Temperature | 20-25° C. |

### INDUSTRIAL APPLICABILITY

A cosmetic of the present invention can provide a cosmetic comprising a nano bubble liquid and cosmetic ingredients, in which the nano bubble liquid is liquid in a nano bubble state containing liquid media and bubbles, the bubbles having a diameter of 5 nm to 500 nm, so that the liquid media and the nano bubble liquid stably contain gas without the risk of gas loss over a long period of time. Furthermore, the cosmetic has a blood flow enhancement effect since the nano bubbles easily permeate into the skin and a cosmetic user can feel warmth after using the cosmetic. Also, the cosmetic has a moisturizing effect, thereby allowing the cosmetic user to keep their skin moist after using the cosmetic. Additionally, since the diameter of the bubble is 5 nm to 500 nm, a cosmetic having no risk of gas loss over a long period of time can be provided.

Accordingly, a cosmetic of the present invention can be suitably and broadly used as a cosmetic having no risk of gas loss over a long period of time and having blood flow enhancement.

1. A cosmetic comprising: a nano bubble-liquid; and cosmetic ingredients, wherein the nano bubble-liquid is liquid in a nano bubble state comprising a liquid medium and bubbles, the bubbles having a diameter of 5 nm to 500 nm.

2. The cosmetic of claim 1, wherein the liquid medium is one or more types of liquid media selected from the group consisting of water and ethanol.

3. The cosmetic of claim 1 or 2, wherein the nano bubble-liquid is in a nano bubble state comprising one or more types of gas selected from the group consisting of hydrogen, oxygen, carbon dioxide and air.

4. The cosmetic of any one of claims 1 to 3, wherein the cosmetic is a skin care cosmetic or a hair cosmetic.

5. The cosmetic of any of claims 1 to 4, wherein the cosmetic is for blood flow enhancement.

6. A method of manufacturing a cosmetic, the method comprising steps of:
(a) obtaining nano bubble-liquid with bubbles having a diameter of 5 nm to 500 nm by supplying gas to a liquid medium; and
(b) blending cosmetic ingredients with a liquid medium,
wherein the steps (a) and (b) are performed in any sequence of simultaneously.

7. The method of claim 6, wherein the liquid medium is one or more types of liquid media selected from the group consisting of water and ethanol.

8. The method of claim 6 or 7, wherein the nano bubbles are made of one or more types of gas selected from the group consisting of hydrogen, oxygen, carbon dioxide and air.

9. The manufacturing method of any one of claims 6 to 8, wherein the cosmetic is a skincare cosmetic or a hair cosmetic.

10. The method of any one of claims 6 to 9, wherein the cosmetic is for blood flow enhancement.
